(19) United States
(12) Patent Application Publication
(10) Pub. No.: US 2019/0249202 A1
(43) Pub. Date: Aug. 15, 2019

(54) Construction of a Lactobacillus Casei Ethanologen

(71) Applicant: Wisconsin Alumni Research Foundation, Madison, WI (US)

(72) Inventors: James L. Steele, Madison, WI (US); Jeff R. Broadbent, Amalga, UT (US)

(21) Appl. No.: 16/394,114

(22) Filed: Apr. 25, 2019

Related U.S. Application Data
(63) Continuation of application No. 13/964,548, filed on Aug. 12, 2013, now abandoned.
(60) Provisional application No. 61/682,281, filed on Aug. 12, 2012.

(51) Int. Cl. C12P 7/06; C12N 9/88; C12N 9/04

(52) U.S. Cl. CPC C12P 7/065; C12N 9/88; C12Y 101/01027; Y02E 50/17; C12N 9/0006; C12Y 401/01001; C12Y 101/01001

(57) ABSTRACT

An engineered bacterium for producing ethanol from one or more carbohydrates is disclosed. The bacterium can be made by (a) inactivating within a Lactobacillus casei bacterium one or more endogenous genes encoding a lactate dehydrogenase; or (b) introducing into a Lactobacillus casei bacterium one or more exogenous genes encoding a pyruvate decarboxylase and one or more exogenous genes encoding an alcohol dehydrogenase II; or (c) performing both steps (a) and (b). The resulting engineered bacterium produces significantly more ethanol than the wild-type Lactobacillus casei bacterium, and can be used in producing ethanol from a substrate such as biomass that includes carbohydrates.

### CROSS-REFERENCE TO RELATED APPLICATIONS

This application is a continuation of U.S. application Ser. No. 13/964,548 filed on Aug. 12, 2013, which claims the benefit of U.S. provisional Application No. 61/682,281 filed on Aug. 12, 2012. Each of these applications is incorporated by reference herein in its entirety.

### STATEMENT REGARDING FEDERALLY SPONSORED RESEARCH OR DEVELOPMENT

This invention was made with government support under DE-FC02-07ER64494 awarded by the US Department of Energy and 2011-67009-30043 awarded by the USDA/NIFA. The government has certain rights in the invention.

### FIELD OF THE INVENTION

This disclosure relates generally to a synthetic Lactobacillus casei bacterium engineered to produce increased amounts of ethanol, as compared to wild type Lactobacillus casei, as well as to methods of making and using such a bacterium.

### BACKGROUND OF THE INVENTION

Microbial production of biofuels from lignocellulosic substrates is a component of the United States plan to reduce its dependency on fossil fuels. The microorganisms typically considered for the production of biofuels include Saccharomyces cerevisiae, Zymomonas mobilis, Escherichia coli, and Clostridium sp. However, all of these microorganisms suffer from one or more of the following deficiencies: relatively low tolerance to the environmental stresses likely to be encountered in fermentation (e.g., high levels of alcohols, acids, and/or osmolarity), complex physiology, poor availability of genetic tools, and limited ability to secrete enzymes. Accordingly, there is a need in the art for improved microorganisms for the production of biofuels such as ethanol from lignocellulosic substrates.

### BRIEF SUMMARY OF THE INVENTION

In a first aspect, this disclosure encompasses an engineered bacterium for producing ethanol from one or more carbohydrates. The engineered bacterium is made by (a) inactivating within a Lactobacillus casei bacterium one or more endogenous genes encoding a lactate dehydrogenase; or (b) introducing into a Lactobacillus casei bacterium one or more exogenous genes encoding a pyruvate decarboxylase and one or more exogenous genes encoding an alcohol dehydrogenase II. The engineered bacterium can also be made using a combination of both approaches. The resulting engineered bacterium produces significantly more ethanol than the wild type Lactobacillus casei bacterium.

In certain embodiments, the Lactobacillus casei bacterium is made from L. casei strain 12A.

In certain embodiments, the step of inactivating within a Lactobacillus casei bacterium one or more endogenous genes encoding a lactate dehydrogenase also includes inactivating within the Lactobacillus casei bacterium an endogenous gene encoding D-hydroxyisocaproate dehydrogenase. In certain embodiments, the engineered bacterium includes the gene deletion mutation ΔL-lactate dehydrogenase 1 (ΔL-ldh1), the gene deletion mutation ΔL-lactate dehydrogenase 2 (ΔL-ldh2), or both. In some such embodiments, the engineered bacterium further includes the gene deletion mutation A D-lactate dehydrogenase (ΔD-ldh) or ΔD-hydroxyisocaproate dehydrogenase (ΔD-hic).

In certain embodiments, the exogenous gene encoding a pyruvate decarboxylase includes the gene of Zymomonas mobilis that encodes for pyruvate decarboxylase (Pdc), and the exogenous gene encoding an alcohol dehydrogenase II includes the gene of Zymomonas mobilis that encodes for dehydrogenase II (AdhII). Preferably, the exogenous genes are modified to utilize L. casei codon usage for highly expressed genes.

In certain embodiments, the exogenous genes are introduced into the L. casei bacterium using an expression vector. A non-limiting example of an expression vector that could be used is pP_{pgm}-PET.

In certain embodiments, the exogenous genes are operably linked to a promoter. Preferably, the promoter is an L. casei promoter. An non-limiting example of a preferred L. casei promoter is the phosphoglycerate mutase (pgm) promoter (P_{pgm}). The L. casei promoter may also be a promoter that is highly expressed in the stationary phase. Non-limiting examples of such promoters include the L. casei GroEL promoter and the the L. casei DnaK promoter.

In a second aspect, the disclosure encompasses an engineered bacterium for producing ethanol from one or more carbohydrates. The engineered bacterium is a derivative of L. casei 12A containing the deletion mutation ΔL-ldh1, an exogenous gene encoding a pyruvate decarboxylase, and an exogenous gene encoding an alcohol dehydrogenase II. The exogenous genes are operably linked to a native L. casei promoter, and the engineered bacterium produces significantly more ethanol than the wild-type L. casei bacterium.

In certain embodiments, the engineered bacterium further includes the deletion mutation ΔL-ldh2.

Non-limiting examples of native L. casei promoters that could be operably linked to the exogenous genes include the phosphoglycerate mutase promoter, the GroEL promoter, and the DnaK promoter.

In certain embodiments, the exogenous genes are from Zymomonas mobilis.

In certain embodiments, the exogenous genes are included in a pP_{pgm}-PET expression vector. In some such embodiments, the pgm promoter (P_{pgm}) in the pP_{pgm}-PET expression vector may be substituted with a promoter that is highly expressed in the stationary phase. Non-limiting examples of such a promoter include a GroEL promoter or a DnaK promoter.

In a third aspect, this disclosure encompasses a method of making ethanol. The method includes the step of culturing the engineered bacterium of any of the embodiments described above on a substrate comprising a carbohydrate, and collecting the ethanol produced by the bacterium.

### BRIEF DESCRIPTION OF THE DRAWINGS

FIG. 1. Growth (▪,●) and glucose utilization (□,∘) by Lactobacillus casei 12AΔ-ldh(pP_{PGM}-PET) (squares) and 12AΔL-IdhlΔL-ldh2ΔD-hic(pP_{PGM}-PET) (circles) at 37° C. in modified chemical defined media (mCDM; Diaz-Muñiz and Steele, 2006) containing 10% glucose (w/v) with pH maintained at 6.0. See Diaz-Muñiz, I. and J. L. Steele, Antonie van Leeuwenhoek 90 (2006): 233-243.

FIGS. 2A and 2B. Growth, glucose consumption, and ethanol production by Lactobacillus casei 12AΔLldh1(pP_{PGM}-PET) (FIG. 2A) and 12AΔL-ldh1ΔL-ldh2ΔD-hic (pP_{PGM}-PET) (FIG. 2B) at 37° C. in a chemically defined media containing 10% glucose with pH maintained at 6.0.

FIG. 3. Metabolism of pyruvate (PYR) in Lactobacillus casei 12A and derivatives. The pyruvate related enzymes and pathways present in L. casei 12A: L-lactate dehydrogenases (L-Ldh); D-lactate dehydrogenase (D-Ldh); D-Hydroxyisocaproate dehydrogenase (D-Hic); acetolactate synthase (Als); oxaloacetate decarboxylase (Oad); pyruvate kinase (Pyk); phophoenolpyruvate carboxikinase (Pck); pyruvate-formate lyase (Pfl); alcohol dehydrogenase (Adh). The enzymes and pathway from Zymomonas mobilis are shown as thick arrows: pyruvate decarboxylase (Pdc); alcohol dehydrogenase (Adh). Abbreviations: EMP, Embden-Meyerof-Parnas pathway; Glu, glucose; PEP, phosphoenolpyruvate.

FIG. 4. Schematic illustrating the gene replacement procedure developed for gene replacement in L. casei 12A. Presence of the pheS* loci results in sensitivity to p-Cl-Phe. This phenotype (derivatives with pheS* form smaller colonies) allows for selection derivatives that have undergone recombination resulting in loss of the pheS* loci (derivatives without phe* form bigger colonies).

FIGS. 5A and 5B. “Production of ethanol” or PET cassette in pTRKH2 designed for L casei 12A, called pP_{pgm}-PET. 5A, Construction of PET cassette in pTRKH2. PET cassette sequence was obtained from Zymomonas mobilis. Codon usage of pdc and adhII were optimized specifically for L. casei 12A using Java Codon Adaptation Tool (Jcat). Codon optimized cassette was synthesized then cloned into pTRKH2 for expression in L casei 12A. 5B, detail of gene organization in the PET cassette: Ppgm, native promoter from L. casei 12A phosphoglycerate mutase; ribosomal binding site (RBS); pdc, pyruvate decarboxylase; adhII, alcohol dehydrogenase; Pin structure, native L. casei 12A transcriptional terminator of kdgR transcriptional regulator protein. The cassette was flanked by PstI and BamHI restriction sites for cloning into pTRKH2.

FIG. 6. Growth curves of L. casei 12A and 12A Δldh1 transformed with empty pTRKH2 (control) or pPgm-PET growth in chemically defined medium (CDM) for 48 hrs. Working cultures were prepared from frozen stocks by two sequential transfers in MRS broth (see J. C. de Man, M. Rogosa and M. Elisabeth Sharpe, Appl. Bact. 23. 130-135 (1960)) with incubations conducted statically at 37° C. for 24 hrs and 18 hrs, respectively. These cultures were then transferred to mCDM overnight and monitored every 6 hrs for OD600 (optical density at 600 nm).

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed nonprovisional applications.

As used herein and in the appended claims, the singular forms “a”, “an”, and “the” include plural reference unless the context clearly dictates otherwise. As well, the terms “a” (or “an”), “one or more” and “at least one” can be used interchangeably herein. The terms “comprising”, “including”, and “having” can be used interchangeably.

Unless defined otherwise, all technical and scientific terms used herein have the same meanings as commonly understood by one of ordinary skill in the art. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

We have developed a bioengineered biofuel-producing strain of Lactobacillus casei. The following characteristics make L. casei an ideal biofuels fermentation organism: ability to use lignocellulosic-derived mono- and di-saccharides; resistance to environmental stresses likely to be encountered in industrial biofuels fermentations, including high levels of biofuels, acids, and/or osmolarity; relatively simple fermentative metabolism with almost complete separation of cellular processes for biosynthesis and energy metabolism; possibility to direct metabolic flux of both pentoses and hexoses to pyruvate (allowing for construction of derivatives producing second generation biofuels (i.e. isobutanol)); the availability of established platforms for introducing and expressing foreign DNA; availability of a deep portfolio of molecular-genetic data related to L. casei ecological adaptation, genomics, transcriptomics, lipidomics, and metabolomics; the ability to secrete and display proteins, hence potential for use in consolidated bioprocessing; and designation as a GRAS (Generally Regarded As Safe) species.

L. casei 12A, a strain isolated from corn silage on the University of Wisconsin-Madison campus, was selected as the biofuels-producing parental strain, due to its alcohol resistance, carbohydrate utilization profile, and amenability to genetic manipulation.

A two pronged approach has been employed to redirect metabolic flux in L. casei 12A to ethanol. The first approach was to inactivate genes that encode enzymes which compete with the 12A pathway to ethanol. The second approach utilized the introduction of the genes from Zymomonas mobilis that encode pyruvate decarboxylase (Pdc) and alcohol dehydrogenase II (Adh2) activities (PET cassette). These genes were designed utilizing the L. casei codon usage for highly expressed genes with a constitutive L. casei promoter (phosphoglycerate mutase), synthesized, ligated with digested pTRKH2 to form pP_{PGM}-PET), and introduced into 12A derivatives by electroporation. This two pronged approach has resulted in an L. casei 12A derivative that produces ethanol as more than 80% of its metabolic end products.

The constructed derivative of L. casei 12A produces ethanol as more than 80% of its final metabolic end products from glucose, and the path to greater than 90% conversion is clear. This is by far the greatest conversion that has been reported with a lactobacilli, and will allow us to exploit the advantages of the use of lactobacilli as biocatalysts for the production of biofuels. These advantages are further delineated below.

The specific features and advantages of the present invention will become apparent after a review of the following experimental examples. However, the invention is not limited to the specific embodiments disclosed herein.

### III. Examples

### Example A

This example addresses (1) what level of carbohydrate Lactobacillus casei 12A derivatives are capable of using; and (2) what level of ethanol production takes place at elevated glucose concentrations.

In the first experiment, 48 small volume (2 ml) fermentations were conducted in GC vials containing our L. casei chemically defined media to examine glucose utilization and end product formation. In parallel, these fermentations were conducted in a 96 well plate reader to monitor growth. The experimental matrix was: 3 levels of glucose (2.5, 5.0, and 10% w/v), with and without the osmoprotectants present in ACSH (0.7 mM betaine, 0.7 mM choline chloride, and 0.2 mMDL-carnitine), with and without 2.5 μg/ml erythromycin (Ery) to select for the plasmid encoded PET cassette, and four different strains. The strains utilized were: (1) an L. casei 12A derivative (12AΔL-ldh1) lacking L-lactate dehydrogenase 1 (L-ldh1), the primary fermentative lactate dehydrogenase, with pTRKH2 (empty vector control); (2) 12AΔL-ldh1 containing pPPGMPET, pTRKH2 with an insert containing the L. casei codon optimized Zymomonas mobilis genes encoding pyruvate decarboxylase (Pdc) and alcohol dehydrogenase II (Adh2) activities under the control of the L. casei phosphoglycerate mutase (pgm) promoter; (3) an L. casei 12 A derivative (12AΔL-ldh1 ΔL-ldh2ΔD-hic) lacking L-ldh1, L-ldh2, and D-hydroxyisocaproate dehydrogenase (D-Hic) containing pTRKH2; and (4) 12AΔL-ldh1ΔL-ldh2ΔD-hic containing pPPGM-PET. These fermentations were conducted at 37° C. for 96 h and the media had an initial pH of 6.0.

Three of the strains (12AΔL-ldh1(pTRKH2), 12AΔL-ldh(pP_{PGM}-PET) and 12AΔL-ldh1ΔLldh2ΔD-hic (pP_{PGM}-PET) reached an OD600 of greater than 1.0 within 24 h and grew at indistinguishable rates regardless of the glucose concentration, the presence or absence of either osmoprotectants, or Ery. The other strain, 12AΔL-ldh1ΔL-ldh2ΔD-hic (pTRKH2) grew poorly, never reaching an OD600 of greater than 0.05, even after 96 h, regardless of media composition; this corresponds with previous experiments and was expected, as this strain lacks an efficient mechanism to regenerate NAD+ from pyruvate.

The addition of osmoprotectants did not have a significant effect on growth of any of the strains under the conditions examined; however, the presence of the osmoprotectants did result in a reduction in lysis of strains producing ethanol in the presence of 2.5% glucose. No lysis was observed by the ethanol producing strains at the higher glucose concentrations, suggesting that the higher osmolarities induced genes that provide enhanced ethanol tolerance. The most significant finding from the growth experiments is that growth of L. casei 12A derivatives is not affected by the glucose (osmolarity) concentrations up to 10%, rather these conditions seem to enhance cell viability in stationary phase of 12A derivatives producing ethanol.

Metabolic end product accumulation in the small volume fermentations were determined by GLBRC Enabling Technologies (HPLC-RID), and the results for L. casei 12AΔL-ldh(pPPGMPET) and 12AΔL-ldh1ΔL-ldh2ΔD-hic(pP_{PGM}-PET) are presented in Table 1. All of the glucose was consumed in fermentations containing 2.5% (139 mM) and 5.0% (278 mM) glucose. In fermentations containing 10% (566 mM) glucose, glucose utilization ranged from 8.1 to 9.5% (459.1 to 536.4 mM). The ethanol formed in the 2.5% (139 mM) glucose fermentations ranged from 1.3 to 1.4% (219.6 to 247.6 mM), with % theoretical yields ranging from 79 to 89%. The ethanol formed in the 5.0% (278 mM) glucose fermentations ranged from 2.6 to 2.7% (438.0 to 466.0 mM), with % theoretical yields ranging from 79 to 84%. The ethanol formed in the 10% (566 mM) glucose fermentations ranged from 3.3 to 3.8% (563 to 651.5 mM), with % theoretical yields ranging from 50 to 58%. In fermentations containing 10% (556 mM) glucose, significant accumulation of pyruvate (73.2 to 92.4 mM) was observed, suggesting that pyruvate decarboxylase activity has become limiting. Under all the conditions examined, L. casei 12AΔLldh1ΔL-ldh2ΔD-hic (pP_{PGM}-PET) produced slightly more ethanol and slightly less lactate than L. casei 12AΔl-ldh (pP_{PGM}-PET). Possible reasons for incomplete glucose utilization in fermentations containing 10% glucose include changes in the pH of the media and increases in pressure due to conducting the fermentations in closed vials. To overcome these issues, fermentations that allow for pH control and CO₂ release have been conducted.

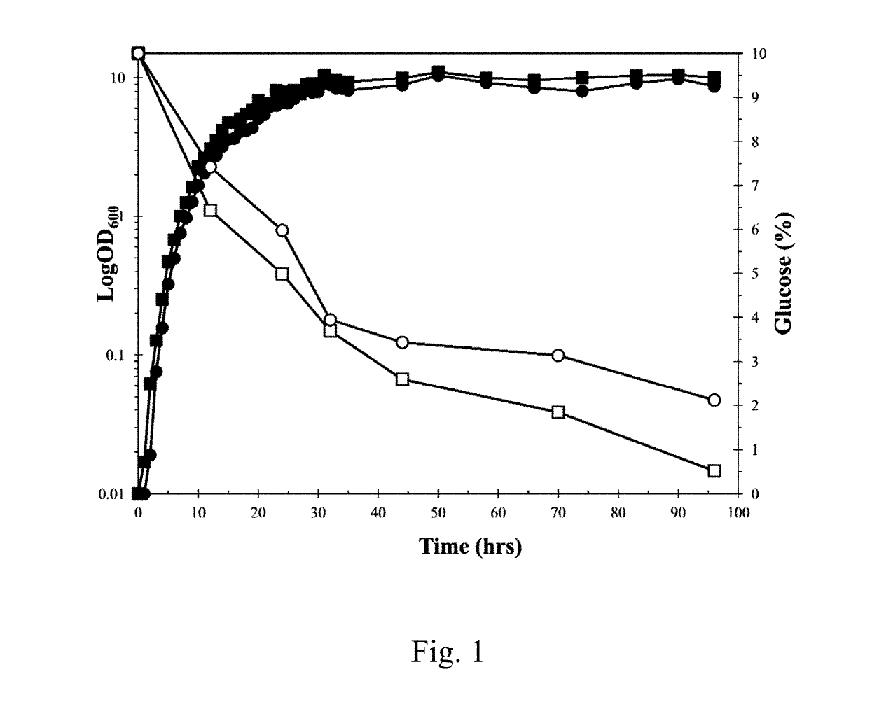

Fermentations with 10% glucose with osmoprotectants and Ery have been conducted in our larger scale (500 ml) fermentation equipment that allows for pH control and CO₂ release with L. casei 12AΔL-ldh (pP_{PGM}-PET) and 12AΔL-ldh1ΔL-ldh2ΔD-hic (pP_{PGM}-PET) at 37° C., with pH maintained at 6.0. The growth and glucose utilization (enzymatic determination) results are presented in FIG. 1. Growth of the two strains are indistinguishable under these conditions; however, greater glucose utilization was observed by 12AΔL-ldh(pP_{PGM}-PET). Metabolic end product accumulation in these fermentations was determined by GLBRC Enabling Technologies (HPLC-RID), and the results are presented in Example B.

The 19 12A derivatives that were constructed via our two-step gene replacement method are presented in Table 2, clearly demonstrating the successful construction of a variety of 12A mutants.

TABLE 1
Metabolic end products accumulated by L. casei 12A ethanologens during growth in a
chemically defined medium (initial pH 6.0) containing a different levels of glucose, with and
without osmoprotectants at 37° C. for 96 hrs.

| | | | | | | | | | | % |
|---|---|---|---|---|---|---|---|---|---|---|
| | Osmo- | Glucose (mM) | | | Products (mM) | | | | Final | Ethanol |
| Strain | protectant | Int | Con. | Rem | EtOH | Pyr | Lac | Ace | pH | (v/v)* |
| 12A ΔL-Ldh1 | − | 142.0 | 142.0 | BQL | 227.9 | BQL | 12.3 | 6.6 | 5.4 | 1.3 |
| (pP_{PGM}-PET) | | | | | | | | | | |
| 12A ΔL-Ldh1 | − | 277.0 | 277.0 | BQL | 438.0 | 14.1 | 26.6 | 4.9 | 4.7 | 2.6 |
| (pP_{PGM}-PET) | | | | | | | | | | |
| 12A ΔL-Ldh1 | − | 499.7 | 393.7 | 106.0 | 600.0 | 73.2 | 31.8 | 5.3 | 4.5 | 3.5 |
| (pP_{PGM}-PET) | | | | | | | | | | |
| 12A ΔL-Ldh1 | + | 137.3 | 137.3 | BQL | 219.6 | BQL | 11.8 | 6.6 | 6.2 | 1.3 |
| (pP_{PGM}-PET) | | | | | | | | | | |
| 12A ΔL-Ldh1 | + | 278.1 | 278.1 | BQL | 445.4 | 3.3 | 39.1 | 3.2 | 4.7 | 2.6 |
| (pP_{PGM}-PET) | | | | | | | | | | |
| 12A ΔL-Ldh1 | + | 499.3 | 469.6 | 29.6 | 563.0 | 86.1 | 40.5 | 5.3 | 4.4 | 3.3 |
| (pP_{PGM}-PET) | | | | | | | | | | |
| 12A ΔL-Ldh1/ | − | 142.3 | 142.3 | BQL | 247.6 | BQL | 7.7 | 8.7 | 7.4 | 1.4 |
| ΔL-Ldh2/ΔD-Hic | | | | | | | | | | |
| (pP_{PGM}-PET) | | | | | | | | | | |
| 12A ΔL-Ldh1/ | − | 282.6 | 282.6 | BQL | 443.5 | 18.3 | 17.9 | 9.1 | 7.2 | 2.6 |
| ΔL-Ldh2/ΔD-Hic | | | | | | | | | | |
| (pP_{PGM}-PET) | | | | | | | | | | |
| 12A ΔL-Ldh1/ | − | 508.0 | 401.1 | 106.9 | 625.0 | 91.6 | 22.3 | 11.7 | 6.6 | 3.6 |
| ΔL-Ldh2/ΔD-Hic | | | | | | | | | | |
| (pP_{PGM}-PET) | | | | | | | | | | |
| 12A ΔL-Ldh1/ | + | 139.5 | 139.5 | BQL | 233.2 | BQL | 7.6 | 8.0 | 6.1 | 1.4 |
| ΔL-Ldh2/ΔD-Hic | | | | | | | | | | |
| (pP_{PGM}-PET) | | | | | | | | | | |
| 12A ΔL-Ldh1/ | + | 280.9 | 280.9 | BQL | 466.0 | 18.1 | 15.2 | 8.3 | 6.7 | 2.7 |
| ΔL-Ldh2/ΔD-Hic | | | | | | | | | | |
| (pP_{PGM}-PET) | | | | | | | | | | |
| 12A ΔL-Ldh1/ | + | 507.9 | 408.9 | 99.0 | 651.5 | 92.4 | 26.0 | 12.4 | 6.8 | 3.8 |
| ΔL-Ldh2/ΔD-Hic | | | | | | | | | | |
| (pP_{PGM}-PET) | | | | | | | | | | |

Abbreviations:
BQL = Below Quantitative Level.
Abbr:
Int—initial,
Con—consumed,
Rem—remaining,
EtOH—ethanol,
Pyr—pyruvate,
Lac—Lactate,
Ace—acetate.

TABLE 2
Lactobacillus casei 12A derivatives constructed in the past
10 months in the Steele laboratory via gene replacement.

| Single | Double | Triple | Quadruple |
|---|---|---|---|
| knockouts | knockouts | knockouts | knockouts |
| ΔL-ldh1* | ΔL-ldh1/ΔL-ldh2* | ΔL-ldh1/ΔL-ldh2/ | ΔoadA/Δpck/ |
| ΔL-ldh2* | ΔL-ldh1/ΔL-ldh3 | ΔL-ldh3 | Δpyc/Δfum |
| ΔL-ldh3 | ΔL-ldh1/ΔL-ldh4 | ΔL-ldh1/ΔL-ldh2/ | ΔoadA/Δpck/ |
| ΔL-ldh4 | ΔL-ldh1/ΔD-ldh | ΔL-ldh4 | Δpyc/Δaspal |
| ΔD-ldh | ΔL-ldh1/ΔD-hic | ΔL-ldh1/ΔLldh2/ | |
| ΔD-hic | ΔL-ldh1/Δpck | ΔD-ldh | |
| Δals | ΔoadA/Δpck | ΔL-ldh1/ΔL-ldh2/ | |
| Δald | ΔoadA/Δpyc | ΔD-hic* | |
| Δa/drc | Δpyc/Δpck | ΔoadA/Δpck/Δpyc | |
| ΔoadA | | | |
| Δpyc | | | |
| Δpck | | | |
| Δaspal | | | |

Abbreviations: L-ldh—L-lactate dehydrogenase, D-ldh—D-lactate dehydrogenase, D-hic—D-hydroxyisocaproate dehydrogenase, als—acetolactate synthase, ald—alpha acetolactate decarboxylase, a/drc—acetoin/diacetyl reductase, oad—oxaloacetate decarboxylase, pyc—pyruvate carboxylase, pck—phosphoenolpyruvate carboxikinase, aspal—aspartate-ammonia lyase, fum—fumarase.
Asterisk—derivatives transformed with pP_{PGM}-PET are available.

### Example B

This example shows the analysis of the data we obtained from the fermentations with 10% glucose with osmoprotectants and Ery that were conducted in our larger scale (500 ml) fermentation equipment with Lactobacillus casei 12AΔL-ldh (pP_{PGM}-PET) and 12AΔL-ldh1 ΔLldh2ΔD-hic (pP_{PGM}-PET) at 37° C., with pH maintained at 6.0. We could only accommodate three fermentation vessels at a time. Therefore, only the 12AΔL-ldh (pP_{PGM}-PET) fermentation was conducted in duplicate.

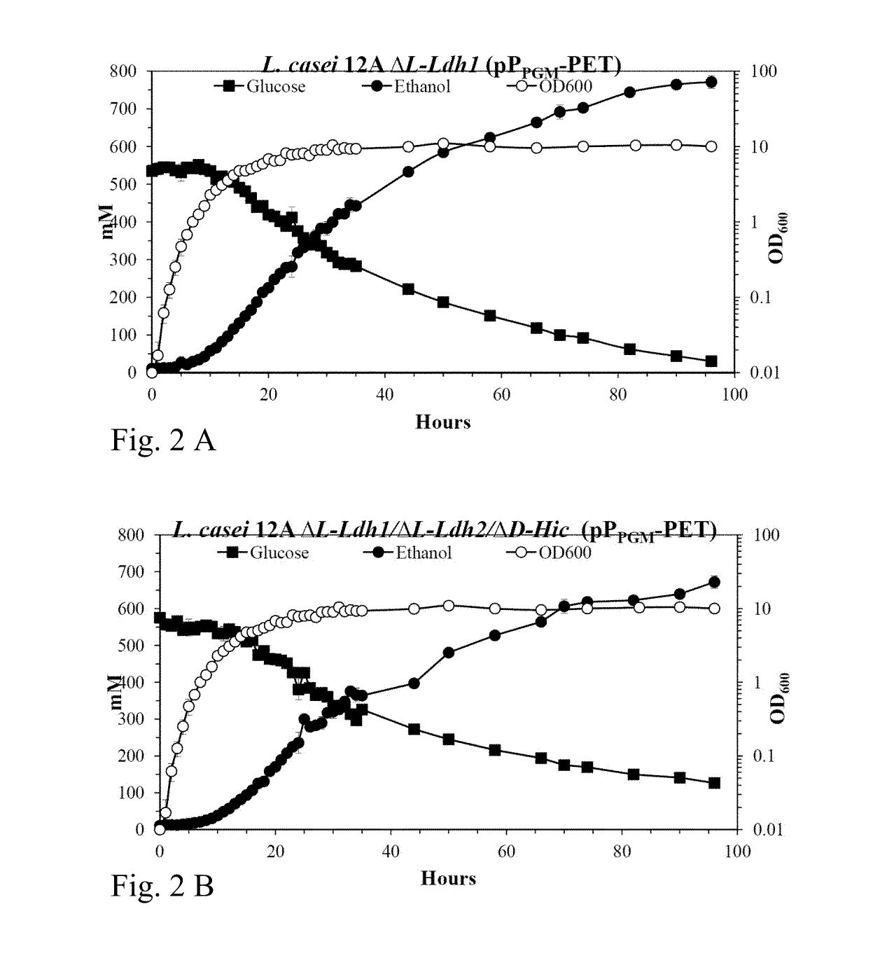

The growth, glucose utilization, and ethanol production shown by these strains are presented in FIGS. 2A (L. casei 12AΔL-ldh (pP_{PGM}-PET)) and 2B (L. casei 12AΔL-ldh1ΔLldh2ΔD-hic (pP_{PGM}-PET)). The growth of the two strains under these conditions was indistinguishable. However 12AΔL-ldh (pP_{PGM}-PET) utilized a greater quantity of glucose and produced more ethanol than 12AΔL-ldh1 ΔL-ldh2ΔD-hic (pP_{PGM}-PET). The glucose utilization and ethanol formation obtained with 12AΔL-ldh (pP_{PGM}-PET) in the larger fermentation vessels was significantly greater than that obtained in the small volume fermentations described in Example A. The mostly likely reason for this difference is that the larger vessels allow for pH control.

The metabolic end products formed and glucose utilized as a function of time for these fermentations is presented in Tables 3 and 4. 12AΔL-ldh1(pP_{PGM}-PET) will be the focus of this discussion, due to its higher productivity. This 12A derivative utilized 504.5 mM glucose (9.1%) glucose in 96 h and produced 934.7 mM of “pyruvate-derived” metabolic end products, which is 87.4% of the theoretical yield from glucose. Ethanol was produced at a level of 771.3 mM (4.5%), which was 82.5% of the metabolic end-products.

The second most abundant metabolic end product was pyruvate, which was present at 110.1 mM after 96 h. Pyruvate accumulation began at approximately 21 h, at the same time, ethanol as a percentage of the total metabolic end products began to decrease (% ethanol in total), suggesting that pyruvate decarboxylase activity becomes limiting at that time. This corresponds to the entry of this organism into stationary phase, suggesting that the L. casei phosphoglycerate mutase (pgm) promoter used to drive expression of the PET cassette is poorly expressed in stationary phase. It is highly likely that pyruvate accumulation can be overcome by utilizing a L. casei promoter highly expressed in stationary phase. If the pyruvate, which had accumulated after 96 h in the 12AΔL-ldh1(pP_{PGM}-PET) fermentation, had been converted to ethanol, a total of 881.4 mM (5.14%) ethanol would have been produced. Additionally, the rate of glucose utilization would have been even higher, as pyruvate accumulation is known to inhibit glycolysis.

It is difficult to directly compare our results to what is known concerning other biocatalysts, due to differences in media and fermentation equipment utilized. However, the results obtained in these L. casei 12AΔL-ldh1(pP_{PGM}-PET) fermentations are most similar to the Escherichia coli GLBRCE1 synthetic hydrolysate fermentations reported by Schwalbach et al. (2012, AEM 78:3442) in E. coli. GLBRCE1 converted 338 mM glucose into 477 mM ethanol, an ethanol yield of 70.5% of the theoretical maximum. L. casei 12AΔL-ldh1(pP_{PGM}-PET) converted 504.5 mM glucose into 771.3 mM ethanol, an ethanol yield of 76.4% of the theoretical maximum.

TABLE 3
Metabolic end products formed and glucose consumption by Lactobacillus casei 12A
ΔL-Ldh1 (pP_{PGM}-PET) at 37° C. in a chemically defined media containing 10% glucose with pH
maintained at 6.0.

| | Glucose | | | | | | | | % | % | Ethanol:Lactate |
|---|---|---|---|---|---|---|---|---|---|---|---|
| Time | (mM) | | Products (mM) | | | | | % | Ethanol | Ethanol | ratio |
| (hr) | Rem | Con | Total | EtOH | Pyr | Lac | Ace | yield | in total | (v/v) | (mM:mM) |
| 0 | 534.9 | BQL | 12.5 | 11.1 | 0.0 | 0.1 | 1.2 | 1.2 | 89.2 | 0.1 | 85 |
| 1 | 540.6 | BQL | 22.7 | 12.0 | 0.0 | 5.4 | 5.3 | 2.1 | 52.7 | 0.1 | 2 |
| 2 | 545.2 | BQL | 24.0 | 12.8 | 0.0 | 5.5 | 5.7 | 2.2 | 53.4 | 0.1 | 2 |
| 3 | 544.8 | BQL | 19.2 | 12.8 | 0.0 | 2.5 | 3.8 | 1.8 | 67.0 | 0.1 | 5 |
| 4 | 536.5 | BQL | 21.5 | 15.0 | 0.0 | 2.3 | 4.2 | 2.0 | 70.0 | 0.1 | 7 |
| 5 | 531.4 | BQL | 33.5 | 27.1 | 0.0 | 1.3 | 5.0 | 3.1 | 81.0 | 0.2 | 20 |
| 6 | 544.4 | BQL | 28.2 | 21.7 | 0.0 | 0.9 | 5.6 | 2.6 | 77.0 | 0.1 | 25 |
| 7 | 542.2 | BQL | 36.8 | 29.4 | 0.0 | 1.8 | 5.7 | 3.4 | 79.8 | 0.2 | 17 |
| 8 | 551.2 | BQL | 42.3 | 34.4 | 0.0 | 2.1 | 5.8 | 4.0 | 81.3 | 0.2 | 16 |
| 9 | 539.9 | BQL | 51.2 | 42.8 | 0.0 | 2.7 | 5.8 | 4.8 | 83.5 | 0.2 | 16 |
| 10 | 535.1 | BQL | 67.0 | 58.1 | 0.0 | 3.3 | 5.6 | 6.3 | 86.7 | 0.3 | 18 |
| 11 | 513.9 | 21.0 | 74.9 | 66.1 | 0.0 | 3.6 | 5.2 | 7.0 | 88.2 | 0.4 | 18 |
| 12 | 521.1 | 13.8 | 90.6 | 82.5 | 0.0 | 2.9 | 5.2 | 8.5 | 91.0 | 0.5 | 28 |
| 13 | 512.8 | 22.1 | 105.1 | 96.8 | 0.0 | 3.4 | 4.9 | 9.8 | 92.1 | 0.6 | 29 |
| 14 | 506.3 | 28.7 | 124.5 | 116.1 | 0.0 | 3.9 | 4.4 | 11.6 | 93.3 | 0.7 | 29 |
| 15 | 490.8 | 44.1 | 140.0 | 131.7 | 0.0 | 4.4 | 4.0 | 13.1 | 94.0 | 0.8 | 30 |
| 16 | 481.5 | 53.5 | 158.7 | 150.1 | 0.0 | 4.9 | 3.7 | 14.8 | 94.6 | 0.9 | 31 |
| 17 | 463.4 | 71.5 | 175.4 | 166.6 | 0.0 | 5.4 | 3.4 | 16.4 | 95.0 | 1.0 | 31 |
| 18 | 439.5 | 95.4 | 196.7 | 187.6 | 0.0 | 5.9 | 3.2 | 18.4 | 95.4 | 1.1 | 32 |
| 19 | 442.9 | 92.0 | 223.0 | 213.2 | 0.0 | 6.7 | 3.1 | 20.8 | 95.6 | 1.2 | 32 |
| 20 | 419.4 | 115.5 | 235.4 | 225.5 | 0.0 | 7.0 | 2.8 | 22.0 | 95.8 | 1.3 | 32 |
| 21 | 414.8 | 120.1 | 258.5 | 247.6 | 0.4 | 7.8 | 2.7 | 24.2 | 95.8 | 1.4 | 32 |
| 22 | 402.2 | 132.7 | 275.1 | 263.0 | 1.1 | 8.4 | 2.7 | 25.7 | 95.6 | 1.5 | 31 |
| 23 | 389.1 | 145.8 | 292.6 | 278.7 | 2.1 | 9.2 | 2.5 | 27.3 | 95.3 | 1.6 | 30 |
| 24 | 412.0 | 122.9 | 297.6 | 281.5 | 3.4 | 9.8 | 2.8 | 27.8 | 94.6 | 1.6 | 29 |
| 25 | 375.9 | 159.0 | 336.1 | 318.4 | 4.4 | 10.8 | 2.5 | 31.4 | 94.7 | 1.9 | 29 |
| 26 | 357.3 | 177.6 | 353.2 | 332.4 | 5.7 | 11.8 | 3.3 | 33.0 | 94.1 | 1.9 | 28 |
| 27 | 343.8 | 191.1 | 363.0 | 339.7 | 7.8 | 12.4 | 3.1 | 33.9 | 93.6 | 2.0 | 27 |
| 28 | 339.7 | 195.2 | 387.7 | 362.0 | 9.4 | 13.3 | 2.9 | 36.2 | 93.4 | 2.1 | 27 |
| 29 | 336.6 | 198.3 | 411.0 | 382.9 | 10.5 | 14.3 | 3.2 | 38.4 | 93.2 | 2.2 | 27 |
| 30 | 318.4 | 216.5 | 411.7 | 383.1 | 11.1 | 14.7 | 2.9 | 38.5 | 93.0 | 2.2 | 26 |
| 32 | 292.4 | 242.5 | 451.8 | 421.5 | 12.9 | 15.1 | 2.3 | 42.2 | 93.3 | 2.5 | 28 |
| 34 | 289.7 | 245.2 | 481.8 | 445.1 | 16.3 | 17.4 | 2.9 | 45.0 | 92.4 | 2.6 | 26 |
| 44 | 221.6 | 313.3 | 588.7 | 533.1 | 29.3 | 22.8 | 3.4 | 55.0 | 90.6 | 3.1 | 23 |
| 50 | 187.2 | 347.7 | 656.5 | 584.7 | 41.6 | 25.7 | 4.4 | 61.4 | 89.1 | 3.4 | 23 |
| 58 | 151.4 | 383.5 | 714.0 | 623.4 | 56.3 | 28.5 | 5.8 | 66.7 | 87.3 | 3.6 | 22 |
| 66 | 118.6 | 416.3 | 768.6 | 664.1 | 65.3 | 31.5 | 7.7 | 71.8 | 86.4 | 3.9 | 21 |
| 70 | 99.9 | 435.0 | 813.2 | 691.8 | 78.8 | 33.4 | 9.2 | 76.0 | 85.1 | 4.0 | 21 |
| 74 | 92.2 | 442.7 | 827.3 | 702.8 | 81.3 | 33.8 | 9.4 | 77.3 | 85.0 | 4.1 | 21 |
| 82 | 62.4 | 472.5 | 873.8 | 744.0 | 81.1 | 36.8 | 11.9 | 81.7 | 85.1 | 4.3 | 20 |
| 90 | 44.1 | 490.8 | 913.8 | 764.4 | 97.8 | 38.3 | 13.2 | 85.4 | 83.7 | 4.5 | 20 |
| 96 | 30.4 | 504.5 | 934.7 | 771.3 | 110.1 | 39.2 | 14.1 | 87.4 | 82.5 | 4.5 | 20 |

TABLE 4
Metabolic end products formed and glucose consumption by Lactobacillus casei 12A
ΔL-Ldh1/ΔL-Ldh2/ΔD-Hic (pP_{PGM}-PET) at 37° C. in a chemically defined media containing 10%
glucose with pH maintained at 6.0.

| | | | | | | | | | % | | |
|---|---|---|---|---|---|---|---|---|---|---|---|
| | Glucose | | | | | | | | Ethanol | % | Ethanol:Lactate |
| Time | (mM) | | Products (mM) | | | | | % | in total | Ethanol | ratio |
| (hr) | Rem | Con | Total | EtOH | Pyr | Lac | Ace | yield | product | (v/v) | (mM:mM) |
| 0 | 575.3 | BQL | 13.3 | 13.3 | 0.0 | 0.0 | 0.0 | 1.2 | 100.0 | 0.1 | — |
| 1 | 557.1 | 18.3 | 12.9 | 12.9 | 0.0 | 0.0 | 0.0 | 1.1 | 100.0 | 0.1 | — |
| 2 | 553.1 | 22.3 | 12.7 | 12.5 | 0.0 | 0.0 | 0.2 | 1.1 | 98.1 | 0.1 | — |
| 3 | 566.0 | 9.3 | 15.2 | 14.5 | 0.0 | 0.0 | 0.6 | 1.3 | 96.0 | 0.1 | — |
| 4 | 541.8 | 33.6 | 16.7 | 15.2 | 0.0 | 0.0 | 1.5 | 1.5 | 90.9 | 0.1 | — |
| 5 | 548.8 | 26.5 | 21.7 | 18.7 | 0.0 | 0.0 | 3.0 | 1.9 | 86.2 | 0.1 | — |
| 6 | 543.2 | 32.1 | 25.8 | 21.0 | 0.0 | 0.0 | 4.9 | 2.2 | 81.2 | 0.1 | — |
| 7 | 551.2 | 24.1 | 31.6 | 25.5 | 0.0 | 0.0 | 6.1 | 2.7 | 80.6 | 0.1 | — |
| 8 | 554.9 | 20.4 | 36.8 | 30.6 | 0.0 | 0.0 | 6.2 | 3.2 | 83.1 | 0.2 | — |
| 9 | 551.3 | 24.0 | 45.6 | 38.5 | 0.0 | 1.1 | 6.0 | 4.0 | 84.4 | 0.2 | 36 |
| 10 | 532.7 | 42.6 | 55.5 | 48.7 | 0.0 | 0.9 | 5.9 | 4.8 | 87.6 | 0.3 | 53 |
| 11 | 532.3 | 43.0 | 64.5 | 57.7 | 0.0 | 1.1 | 5.8 | 5.6 | 89.4 | 0.3 | 54 |
| 12 | 544.0 | 31.3 | 76.6 | 70.6 | 0.0 | 0.0 | 6.1 | 6.7 | 92.1 | 0.4 | — |
| 13 | 536.6 | 38.7 | 87.7 | 82.1 | 0.0 | 0.0 | 5.6 | 7.6 | 93.6 | 0.5 | — |
| 14 | 519.6 | 55.8 | 101.5 | 93.7 | 0.0 | 2.6 | 5.2 | 8.8 | 92.3 | 0.5 | 36 |
| 15 | 510.2 | 65.1 | 111.9 | 107.0 | 0.0 | 0.0 | 4.9 | 9.7 | 95.7 | 0.6 | — |
| 16 | 513.9 | 61.4 | 134.2 | 125.5 | 0.0 | 3.8 | 4.8 | 11.7 | 93.6 | 0.7 | 33 |
| 17 | 474.2 | 101.1 | 134.7 | 130.7 | 0.0 | 0.0 | 3.9 | 11.7 | 97.1 | 0.8 | — |
| 18 | 485.3 | 90.0 | 167.7 | 158.3 | 0.0 | 5.5 | 3.9 | 14.6 | 94.4 | 0.9 | 29 |
| 19 | 463.7 | 111.6 | 179.9 | 170.4 | 0.0 | 5.9 | 3.5 | 15.6 | 94.8 | 1.0 | 29 |
| 20 | 462.1 | 113.2 | 199.4 | 189.4 | 0.0 | 6.8 | 3.2 | 17.3 | 95.0 | 1.1 | 28 |
| 21 | 459.0 | 116.3 | 218.4 | 207.8 | 0.0 | 7.6 | 3.0 | 19.0 | 95.1 | 1.2 | 27 |
| 22 | 451.9 | 123.5 | 236.9 | 224.9 | 0.7 | 8.6 | 2.8 | 20.6 | 94.9 | 1.3 | 26 |
| 23 | 426.3 | 149.0 | 238.0 | 235.8 | 0.0 | 0.0 | 2.3 | 20.7 | 99.0 | 1.4 | — |
| 24 | 380.6 | 194.7 | 314.8 | 299.9 | 3.5 | 9.4 | 1.9 | 27.4 | 95.3 | 1.8 | 32 |
| 25 | 426.0 | 149.3 | 295.3 | 279.1 | 3.6 | 10.4 | 2.2 | 25.7 | 94.5 | 1.6 | 27 |
| 26 | 384.5 | 190.8 | 297.1 | 283.3 | 2.5 | 9.6 | 1.7 | 25.8 | 95.3 | 1.7 | 29 |
| 27 | 365.0 | 210.3 | 301.5 | 289.6 | 0.5 | 9.6 | 1.8 | 26.2 | 96.1 | 1.7 | 30 |
| 28 | 371.3 | 204.1 | 331.6 | 317.2 | 1.7 | 11.0 | 1.6 | 28.8 | 95.7 | 1.9 | 29 |
| 29 | 360.5 | 214.8 | 339.2 | 320.3 | 5.6 | 10.9 | 2.3 | 29.5 | 94.4 | 1.9 | 29 |
| 30 | 332.9 | 242.4 | 341.2 | 326.4 | 2.9 | 10.9 | 1.0 | 29.7 | 95.7 | 1.9 | 30 |
| 32 | 333.5 | 241.8 | 395.1 | 375.6 | 5.8 | 12.5 | 1.2 | 34.3 | 95.1 | 2.2 | 30 |
| 34 | 296.7 | 278.6 | 381.7 | 363.9 | 5.3 | 12.1 | 0.4 | 33.2 | 95.3 | 2.1 | 30 |
| 44 | 272.5 | 302.8 | 520.6 | 480.5 | 22.4 | 14.4 | 3.2 | 45.2 | 92.3 | 2.8 | 33 |
| 50 | 245.1 | 330.2 | 578.6 | 527.3 | 30.5 | 16.6 | 4.2 | 50.3 | 91.1 | 3.1 | 32 |
| 58 | 216.0 | 359.4 | 627.0 | 564.1 | 39.4 | 17.6 | 5.8 | 54.5 | 90.0 | 3.3 | 32 |
| 66 | 194.1 | 381.3 | 681.0 | 606.2 | 49.2 | 18.3 | 7.2 | 59.2 | 89.0 | 3.5 | 33 |
| 70 | 175.2 | 400.1 | 700.5 | 618.2 | 54.8 | 19.5 | 8.0 | 60.9 | 88.3 | 3.6 | 32 |
| 74 | 169.5 | 405.8 | 706.8 | 622.6 | 56.7 | 19.2 | 8.3 | 61.4 | 88.1 | 3.6 | 32 |
| 82 | 149.8 | 425.5 | 712.2 | 639.6 | 63.1 | 0.0 | 9.5 | 61.9 | 89.8 | 3.7 | — |
| 90 | 141.0 | 434.3 | 752.9 | 672.0 | 70.1 | 0.0 | 10.8 | 65.4 | 89.3 | 3.9 | — |
| 96 | 126.6 | 448.7 | 774.2 | 663.3 | 79.0 | 21.1 | 10.7 | 67.3 | 85.7 | 3.9 | 31 |

Abbreviations in Tables: Rem, Remaining; Con, consumed; EtOH, ethanol; Pyr, pyruvate; Lac, lactate; Ace, acetate. % yield=(mM Total product/(2×mM initial Glucose))×100% Ethanol=(mmol/L ethanol×46.068 g/mol)/(1000 mg/g)×(1000 ml/L/100 ml)×(0.789 g/ml).

### Example C

Screening Strains of L. casei for Biofuels Relevant Phenotypes and Genes.

Our laboratory has a culture collection contains approximately 60 strains of L. casei isolated from green plant material (i.e. corn silage), cheese, wine, and humans. The eleven strains with genome sequences were screened for the ability to utilize 60 different carbohydrates, including numerous carbohydrates present in lignocellulosic feed stocks. Individual strains were able to grow on between 17 and 26 different substrates. The strains isolated from corn silage (12A and 32G) grew on the greatest number of substrates. Nine gene clusters potentially involved in cellobiose utilization and one gene cluster involved in xylose utilization were identified.

The eleven strains with genomic information were also screened for alcohol tolerance (ethanol, 1-propanol, 1-butanol, and 2-methyl-1-butanol), growth in AFEX-pretreated corn stover hydrolysate (ACSH), and transformation (electroporation) efficiency. L. casei 12A exhibited the greatest tolerance to the biofuels examined. For example, when grown in the presence of 10% ethanol, it reached a final cell density 40% of that it attained in the absence of ethanol. Of the 11 strains examined for growth in corn stover hydrolysate, 3 of these strains (ATCC 334, 21-1, and 12A) grew significantly better, reaching a final optical density at 600 nm of approximately 2.0 within 28 h. Five L. casei strains were examined for transformation efficiency with pTRKH2 (O'Sullivan and Klaenhammer 1993). L. casei 12A exhibited a frequency (approximately 5×10⁵ transformants per ug of pTRKH2) at least 50-fold higher than that observed with any of the other strains examined. Based upon the results from these analyses, L. casei 12A was selected as the biofuel producing parental strain.

Completing the L. casei 12A Genome.

For further information regarding the L. casei 12A genome, see Broadbent, et al., BMC Genomics 2012, 13:533, which is incorporated by reference herein. To enhance the depth of genomic sequence coverage of 12A, genomic DNA was prepared and submitted to the Joint Genome Institute (JGI) for genome sequencing. A draft genome of L. casei 12A with approximately 500× coverage assembled into 397 scaffolds was received from JGI. This genome assembly was subsequently merged with the previous 23× 454-generated paired end genome assembly in collaboration with personnel from DuPont Inc. (Madison, Wis.), yielding a genome assembly with 19 ordered contigs. We have generated PCR amplicons across all 19 gaps, and have sequenced 10 of these amplicons.

L. casei Metabolic Models.

We have developed a genome-scale metabolic model for L. casei ATCC334 (the neotype strain) and 12A using the ModelSEED database and the genome annotation from RAST. We have modified the draft L. casei 12A model from ModelSEED using the following processes: 1) thermodynamically infeasible cycles were removed, 2) elementally imbalanced metabolic reactions were corrected; and 3) model predictions for amino acid requirements were compared against experimental growth phenotypes determined in a lactobacilli chemically defined medium (CDM) described by Christensen and Steele (J. Bacteriol. 185 (2003): 3297-3306). Inconsistencies were corrected by the addition or deletion of some reactions.

Redirecting Metabolic Flux in L. casei 12A to Ethanol.

The development of a method to inactivate genes in L. casei was a requirement for the construction of a L. casei strain capable of converting lignocellulosic biomass to ethanol. An efficient gene replacement method based on the introduction of pCJK47-based constructs (Kristich et al. 2007) via a 12A optimized electroporation protocol was developed.

A two pronged approach was employed to redirect metabolic flux in L. casei 12A to ethanol. The first approach is to inactivate genes that encode enzymes which compete with the 12A pathway to ethanol, which has acetyl-CoA as an intermediate. There are a large number of genes that encode enzymes potentially involved in anaerobic pyruvate metabolism in L. casei. We have inactivated 9 of these genes: pyruvate-formate lyase (Pfl), the four L-lactate dehydrogenases (L-ldh1, Lldh2, L-ldh3, and L-ldh4), D-lactate dehydrogenase (D-ldh), D-hydroxyisocaproate dehydrogenase (DHic), acetolactate synthase (Als), and oxaloacetate decarboxylase (OadA). Additionally, 5 derivatives lacking two or three of the dehydrogenases have been constructed. Characterization of the end product distribution these mutants is presented in Table 5. The highest level of metabolic redirection to ethanol achieved to date using this approach, is 21%, achieved with the 12A ΔL-ldh1 ΔL-ldh2ΔD-hic derivative. It is interesting to note that this derivative also accumulates pyruvate.

The second approach utilized to direct metabolic flux in 12A towards ethanol was the introduction of the genes from Zymomonas mobilis that encode pyruvate decarboxylase (Pdc) and alcohol dehydrogenase II (Adh2) activities (PET cassette). These genes were designed utilizing the L. casei codon usage for highly expressed genes with a constitutive L. casei promoter (phosphoglycerate mutase), synthesized by GeneArt, ligated with digested pTRKH2 (pPGM-PET), and introduced into 12A derivatives by electroporation. Characterization of the end product distribution of two of these derivatives has been completed and is presented in Table 5. The highest level of metabolic redirection to ethanol achieved to date using this approach is 85.3%, achieved with the 12A ΔL-ldh1 ΔL-ldh2 (pP_{pgm}-PET) derivative. It is interesting to note that 12A derivatives with pP_{pgm}-PET grow more rapidly than their corresponding strains, suggesting that ethanol is less inhibitory to 12A derivatives than lactate.

These results suggest that the two pronged approach is effective for redirecting 12A metabolic flux to ethanol.

TABLE 5
Growth, substrate consumption, and metabolic end products formed by Lactobacillus casei 12A and derivatives
during growth in a chemically defined media at 37° C. for 48 hrs.

| | | | Concentration (mM) | | | | | | | | | |
|---|---|---|---|---|---|---|---|---|---|---|---|---|
| | Growth | | Substrate | | Metabolic End Products | | | | | | | |
| | Max | T_{d} | Utilization^{a} | | (% of total)^{b} | | | | | | Yield | EtOH/Lac |
| Derivative | OD₆₀₀ | (h) | Glc | Cit | Total | L-lac | D-lac | EtOH | Ace | Pyr | (%)^{c} | ratio^{d} |
| 12A | 1.05 | 8.1 | 51.5 | 0.6 | 112.2 | 105.4, | 3.3, | 1.4, | 2.1, | BQL | 108 | 0.0 |
| | | | | | | (94) | (3) | (1) | (2) | | | |
| 12A ΔL-ldh1 | 1.28 | 7.0 | 52.6 | 11.5 | 87.0 | 42.3, | 28.2, | 16.5, | BQL | BQL | 68 | 0.2 |
| | | | | | | (49) | (32) | (19) | | | | |
| 12A ΔL-ldh2 | 1.01 | 8.3 | 53.1 | 5.8 | 111.8 | 105.0, | 3.2, | 1.6, | 2.0, | BQL | 95 | 0.0 |
| | | | | | | (94) | (3) | (1) | (2) | | | |
| 12A ΔL-ldh3 | 1.02 | 8.0 | 52.7 | 9.0 | 110.2 | 103.0, | 3.2, | 2.1, | 1.9, | BQL | 90 | 0.0 |
| | | | | | | (94) | (3) | (2) | (2) | | | |
| 12A ΔD-ldh | 1.02 | 7.7 | 51.9 | BQL | 112.4 | 103.5, | 5.4, | 1.1, | 2.4, | BQL | 108 | 0.0 |
| | | | | | | (92) | (5) | (1) | (2) | | | |
| 12A ΔD-hic | 1.26 | 7.9 | 51.5 | BQL | 112.0 | 109.7, | BQL | 0.5, | 1.8, | BQL | 109 | 0.0 |
| | | | | | | (98) | | (1) | (2) | | | |
| 12A ΔL-ldh1/ | 1.26 | 7.1 | 52.8 | 15.0 | 86.7 | 32.1, | 42.6, | 12.0, | BQL | BQL | 64 | 0.2 |
| ΔD-ldh | | | | | | (37) | (49) | (14) | | | | |
| 12A ΔL-ldh1/ | 1.11 | 9.7 | 51.2 | 13.9 | 79.8 | 64.9, | BQL | 14.9, | BQL | BQL | 61 | 0.2 |
| ΔD-hic | | | | | | (81) | | (19) | | | | |
| 12A ΔL-ldh1/ΔL- | 0.93 | 9.4 | 52.5 | 10.3 | 71.4 | BQL | 51.5, | 18.6, | BQL | 1.3, | 57 | 0.4 |
| ldh2/ΔD-ldh | | | | | | | (72) | (26) | | (2) | | |
| 12A ΔL-ldh/ΔL- | 0.52 | 31.3 | 21.7 | 12.8 | 36.1 | 0.6, | 0.4, | 7.6, | 7.2, | 20.3, | 52 | 7.6 |
| ldh2/ΔD-hic | | | | | | (2) | (1) | (21) | (20) | (56) | | |
| 12A (pTRKH2) | 1.01 | 13.2 | 52.5 | BQL | 108.9 | 100.4, | 7.6, | BQL | 0.9, | BQL | 104 | 0.0 |
| | | | | | | (92) | (7) | | (1) | | | |
| 12A (pPGM-PET) | 0.95 | 6.79 | 51.3 | 8.2 | 95.1 | 14.8, | 13.2, | 58.1, | 9.0, | BQL | 80 | 2.1 |
| | | | | | | (16) | (14) | (61) | (10) | | | |
| 12A ΔL-ldh1 | 1.11 | 11.3 | 52.3 | 2.7 | 87.7 | 41.6, | 34.0, | 12.1, | BQL | BQL | 80 | 0.2 |
| (pTRKH2) | | | | | | (47) | (39) | (14) | | | | |
| 12A ΔL-ldh1 | 1.03 | 6.8 | 51.0 | 16.3 | 102.1 | 2.7, | 5.0, | 84.5, | 9.8, | BQL | 76 | 10.9 |
| (pPGM-PET) | | | | | | (3) | (5) | (83) | (10) | | | |
| 12A ΔL-ldh1/ΔL- | 1.01 | 7.9 | 50.9 | 16.0 | 100.2 | 0.7, | 5.1, | 85.3, | 9.1, | BQL | 75 | 14.7 |
| ldh2 (pPGM-PET) | | | | | | (1) | (5) | (85) | (9) | | | |

^{a}Reported by the initial concentration of glucose or citrate subtracted by the final concentration of the respective compound at 48 hrs.
^{b}In parenthesis, metabolic end product distribution by % of total.
^{c}Calculated by percentage of total metabolic end products produced/2 × (glucose + citrate) in mmoles.
^{d}Expressed as molar ratio, where lactate is the summation of both the L- and D-forms.
Abbreviations:
BQL = below quantifiable level;
NA = not applicable;
Glu = glucose;
Cit = citrate;
Lac = lactate;
ETOH = ethanol;
Ace = acetate;
Pyr = pyruvuate.

### Example D

Conversion of a Lactic Acid Bacterium Lactobacillus casei 12A to an Ethanologen

Lactobacillus casei 12A was selected as the biofuels parental strain based upon its alcohol tolerance (grows in the presence of >10% ethanol), carbohydrate utilization, and relatively high transformation efficiency. This organism metabolizes hexoses through the Embden-Meyerhof-Parnas pathway and converts pyruvate to lactate via a variety of different enzymes; including four L-lactate dehydrogenases (Ldh), one D-Ldh, and one D-hydroxyisocaproate dehydrogenase.

Essential characteristics of organisms to be utilized for microbial production of ethanol from plant biomass include the ability to secrete enzymes, transport glucose and xylose, metabolize glucose and xylose to ethanol, as well as have sufficient ethanol tolerance to make the fermentation economically viable. It is unlikely an organism capable of meeting all of these criteria will be isolated from nature. Therefore, rational strategies to engineer strains for the industrial production of ethanol from plant biomass are preferred. The following characteristics make L. casei 12A an ideal Gram-positive species for research in this area:
Designation as a GRAS (Generally Regarded As Safe) species.
Established platforms for introducing and expressing foreign DNA.
Relatively simple fermentative metabolism with almost complete separation of cellular processes for biosynthesis and energy metabolism.
Resistance to environmental stress, including high concentrations of acids and biofuels
Ability to use lignocellulosic carbohydrates.
Ability to secrete and display proteins, hence potential for use in consolidated bioprocessing.

We pursued two strategies concurrently to redirect L. casei 12A fermentation to ethanol. The first strategy involved inactivation of enzymes that consume pyruvate under anaerobic conditions without producing ethanol, including the D-Ldh; four L-Ldhs; D-(D-Hic); acetolactate synthase (Als); and oxaloacetate decarboxylase (Oad). This approach has been used to inactivate L-ldh1, L-ldh2, and D-hic, as well as to construct the L-ldh1/L-ldh2, double mutant. The highest level of ethanol formation was observed with the ΔL-ldh1/ΔL-ldh2 double mutant, which produces ethanol as 14% of its metabolic end products.

Our second strategy for increasing flux to ethanol involved expressing ethanol producing enzymes. A codon optimized “PET” cassette comprised of the Zymomonas mobilis genes encoding pyruvate decarboxylase (Pdc) and alcohol dehydrogenase (Adh2) was constructed, and placed under the control of the L. casei 12A pgm promoter, pgm ribosomal binding site and kdgR transcriptional terminator. When this construct was introduced into L. casei 12A, ethanol made up 61% of metabolic end products formed. When introduced into L. casei 12A (ΔL-ldh1), ethanol was the dominant product observed (91% of metabolic end productions). Results from this analysis indicate that the two approaches are complementary and demonstrate that redirecting metabolic flux in L. casei from lactate to an alcohol can be readily achieved.

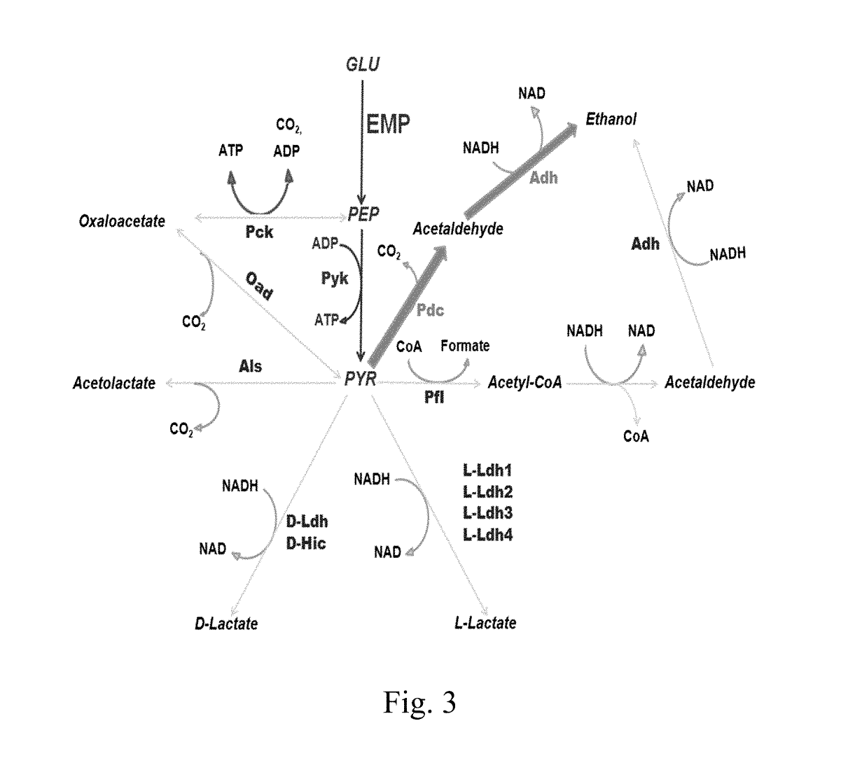

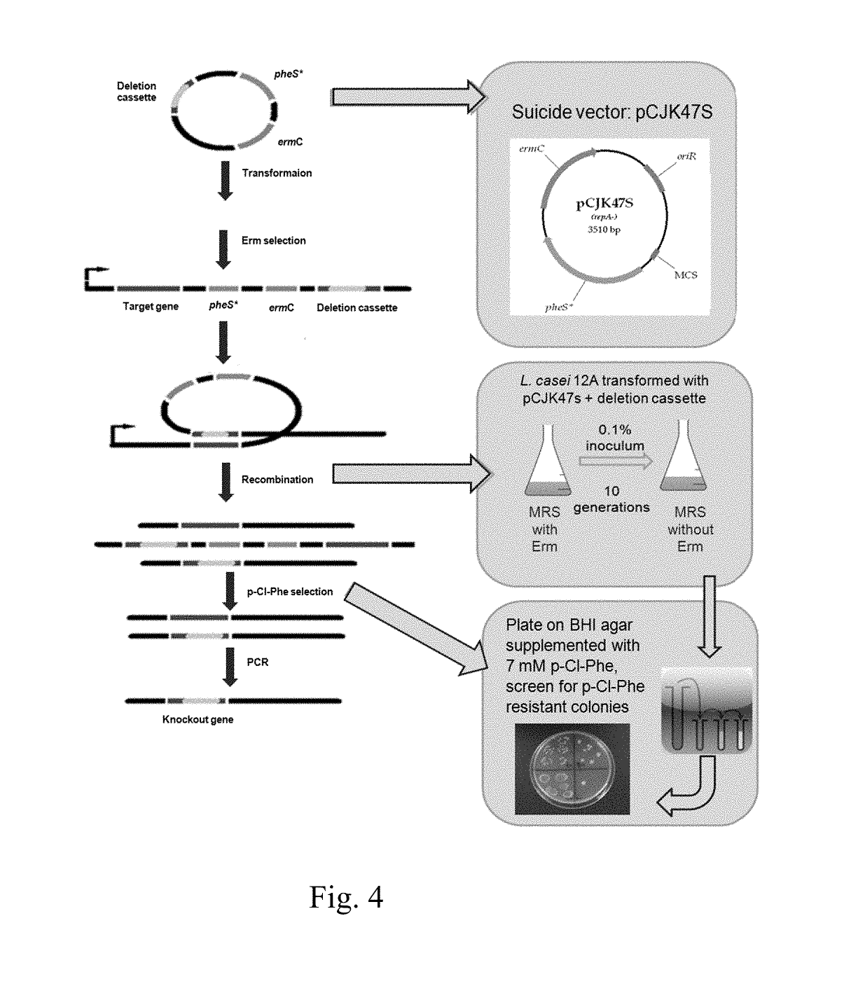

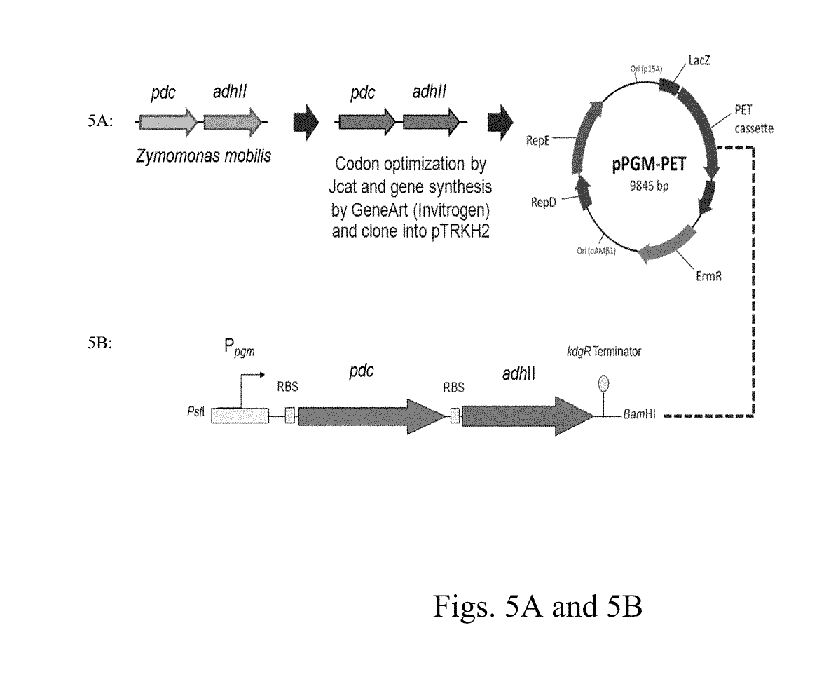

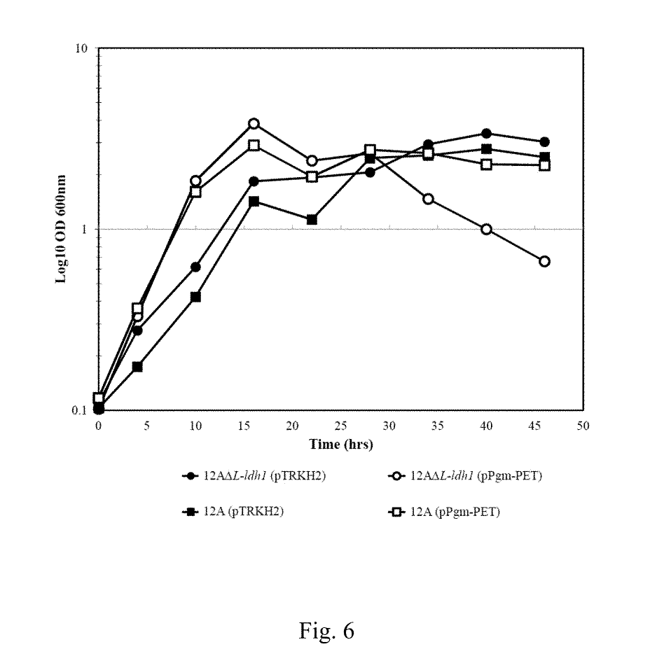

The general strategy that was used to redirect metabolic flux in L. casei 12A from lactic acid to ethanol is illustrated in detail in FIG. 3. Two different methods were used to carry out the strategy. The first method, involving gene deletion, is illustrated in FIG. 4. The second method, involving the construction and subsequent expression of a synthetic PET expression cassette construct in pTRKH2, is illustrated in FIG. 5. The growth of the resulting L. casei 12A ethanologens in Chemically Defined Medium (CDM) is illustrated in FIG. 6. The fermentation by-products of the L. casei mutants grown in CDM were measured, and the results are shown in Table 6.

TABLE 6
Fermentation products of L. casei 12A and mutants with
and without pTRKH2 or pPGM-PET growth in CDM for 48 hrs.

| | Ethanol | L-Lactate | D-Lactate |
|---|---|---|---|
| Derivative | (%) | (%) | (%) |
| 12A | 0.0 | 95.0 | 5.0 |
| 12A ΔL-ldh1 | 6.0 | 49.0 | 45.0 |
| 12A ΔL-ldh2 | 0.0 | 96.0 | 4.0 |
| 12A ΔD-hic | 0.0 | 71.0 | 29.0 |
| 12A ΔL-ldh1ΔL-ldh2 | 14.0 | 34.0 | 52.0 |
| 12A(pTRKH2) | 0.0 | 95.0 | 5.0 |
| 12A (Ppgm-PET) | 61.0 | 34.0 | 1.0 |
| 12A ΔL-ldh1(pTRKH2) | 13.0 | 47.0 | 40.0 |
| 12A ΔL-ldh1(Ppgm-PET) | 90.9 | 1.5 | 1.5 |

Note:
L. casei 12A mutants were grown in MRS from glycerol stock for 24 hrs at 37° C. then transferred to MRS and incubated for an additional 18 hrs. CDM containing 50 mM glucose was inoculated and incubated in GC vials for 48 hrs at 37° C. At the 48-hr time point, supernanant was drawn off and submitted to GLBRC enabling technologies for fermentation by-product analysis via HPLC-RID.

Conclusions.

Inactivation of L-Ldh1 reduced flux towards L-lactate and enhanced flux towards D-lactate and ethanol. Inactivation of L-Ldh2 increased these changes in metabolic flux.

In L. casei 12A with the PET cassette, ethanol made up 61% of metabolic end products formed, while 91% of metabolic end productions were directed to ethanol when the PET cassette was introduced into L. casei 12A ΔL-ldh1.

The two pronged strategy, inactivating genes encoding enzymes that produce lactic acid and introducing the PET cassette, effectively converted L. casei 12A from producing lactate as its main metabolic product to producing ethanol as its main metabolic end product.

### REFERENCES

Cai, H., Thompson, R. L., Broadbent, J. R., and Steele, J. L. (2009). Genome Sequence and Comparative Genome Analysis of Lactobacillus casei: Insights into their Niche-associated Evolution. Genome Biol. and Evol. 1:239-257.
Duong, T., Miller, M. J., Barrangou, R., Azcarate-Peril, M. A., and Klaenhammer, T. R. (2010). Construction of vectors for inducible and constitutive gene expression in Lactobacillus. Microbiol Biotech, 4(3): 357-367.
Kristich, C. J., Chandler, J. R., and Dunny, G. M. (2007). Development of a host-genotype-independent counterselectable marker and a high-frequency conjugative delivery system and their use in genetic analysis of Enterococcus faecalis. Plasmid 57:131-144.

### Example E

### Use of Alternate Promoter

In the previous examples, a first generation Lactobacillus casei ethanologen was created by a two pronged approach to redirect metabolic flux in L. casei 12A from lactate to ethanol. The first prong was to inactivate genes encoding lactate dehydrogenases, enzymes which compete with the 12A pathway to ethanol. The second prong was the introduction of the genes from Zymomonas mobilis that encode pyruvate decarboxylase (Pdc) and alcohol dehydrogenase II (Adh2) activities (PET cassette). These genes were designed utilizing the L. casei codon usage for highly expressed genes and placed under the control of L. casei phosphoglycerate mutase promoter, thought to be a constitutively expressed promoter.

This approach was highly successful, resulting in a strain that utilized 504.5 mM glucose (9.1%) glucose in 96 h and produced 934.7 mM of “pyruvate-derived” metabolic end products, which is 92.6% of the theoretical yield from 504.5 mM glucose in a 500 ml fermentation vessel under anaerobic conditions at 37° C. in a defined media with 540 mM glucose. Ethanol was produced at a level of 771.3 mM (4.5%), which was 82.5% of the metabolic end-products. The second most abundant metabolic end product was pyruvate which was present at 110.1 mM after 96 h.

Pyruvate accumulation began at approximately 21 h. At the same time, ethanol as a percentage of the total metabolic end products began to decrease (% ethanol in total), suggesting that pyruvate decarboxylase activity becomes limiting at that time. This corresponds to the entry of this organism into stationary phase, suggesting that the L. casei phosphoglycerate mutase (pgm) promoter used to drive expression of the PET cassette is poorly expressed in stationary phase. It is highly likely that pyruvate accumulation can be overcome by utilizing a L. casei promoter highly expressed in stationary phase.

Accordingly, this prophetic example discloses the next generation L. casei ethanologen, having the PET cassette placed under the control of a promoter that is highly expressed in stationary phase. For example, either the GroEL or DnaK promoters, as they have been demonstrated to be highly expressed in a related organism, L. plantarum, when this organism was exposed to ethanol (Gyu et al. 2012). The anticipated result from such a construct would be that the 110.1 mM pyruvate that was observed to accumulate in the previous fermentation (see above) would be converted to ethanol. This would then yield 881.4 (110.1+771.3) mM of ethanol, or 87.4% of the theoretical yield from 504.5 mM glucose.

### REFERENCES

Lee, S. G., K. W. Lee, T. H. Park, J. Y. Park, N. S. Han, and J. H. Kim. 2012. Proteomic analysis of proteins increased or reduced by ethanol of Lactobacillus plantarum ST4 isolated from makgeolli, traditional Korean rice wine. J. Microbiol. Biotechnol. 22:516-525.

We claim:

1. A method of making ethanol comprising:
culturing on a substrate comprising a carbohydrate an engineered bacterium comprising a Lactobacillus casei bacterium that includes one or more exogenous genes encoding a pyruvate decarboxylase and one or more exogenous genes encoding an alcohol dehydrogenase II, each of which is operably linked to a L. casei promoter that is highly expressed in the stationary phase, whereby the engineered bacterium produces a composition comprising ethanol.

2. The method of claim 1, wherein the engineered bacterium further includes one or more gene deletion mutations of one or more endogenous genes encoding a lactate dehydrogenase.

3. The engineered bacterium of claim 1, wherein the engineered bacterium further includes a gene deletion mutation of an endogenous gene encoding D-hydroxyisocaproate dehydrogenase.

5. The method of claim 2, wherein the gene deletion mutations comprise Δ L-lactate dehydrogenase 1 (ΔL-ldh1).

5. The method of claim 2, wherein the gene deletion mutations comprise Δ L-lactate dehydrogenase 2 (ΔL-ldh2).

6. The method of claim 1, wherein the engineered bacterium includes the gene deletion mutations Δ D-lactate dehydrogenase (ΔD-ldh) or Δ D-hydroxyisocaproate dehydrogenase (ΔD-hic).

7. The method of claim 1, wherein the exogenous gene encoding a pyruvate decarboxylase comprises the gene of Zymomonas mobilis that encodes for pyruvate decarboxylase (Pdc), and the exogenous gene encoding an alcohol dehydrogenase II comprises the gene of Zymomonas mobilis that encodes for alcohol dehydrogenase II (AdhII).

8. The method of claim 1, wherein the exogenous genes are modified to utilize codon usage for highly expressed genes in L. casei.

9. The method of claim 1, wherein the exogenous genes are introduced into the L. casei bacterium using an expression vector.

10. The method of claim 9, wherein the expression vector is an expression vector comprising the Zymomonas mobilis genes encoding for pyruvate decarboxylase (Pdc) and alcohol dehydrogenase II (Adh2) and a L. casei promoter that is highly expressed in the stationary phase.

11. The method of claim 1, wherein the strain of the Lactobacillus casei bacterium is strain 12A.

12. The method of claim 1, wherein the L. casei promoter that is highly expressed in the stationary phase is the GroEL promoter or the DnaK promoter.

13. A method of claim 1, further comprising collecting the ethanol from the composition produced by the engineered bacterium.

14. The method of claim 1, wherein the amount of ethanol in the composition produced is significantly greater than the amount of ethanol that would be produced by a Lactobacillus casei bacterium cultured on a substantially similar substrate for the same amount of time that is either (a) a wild-type Lactobacillus casei bacterium or (b) a Lactobacillus casei bacterium including one or more exogenous genes encoding a pyruvate decarboxylase and one or more exogenous genes encoding an alcohol dehydrogenase II, each of which is operably linked to a L. casei promoter that is not highly expressed in the stationary phase.

15. The method of claim 1, wherein the engineered bacterium remains cultured on the substrate after the engineered bacterium has entered into the stationary phase.

16. The method of claim 1, wherein the engineered bacterium is cultured on the substrate for greater than 21 hours.

17. The method of claim 15, wherein the amount the ethanol produced when the engineered bacterium is in the stationary phase is significantly greater than the amount of ethanol produced during the stationary phase by a Lactobacillus casei bacterium cultured on a substantially similar substrate for the same amount of time that is either (a) a wild-type Lactobacillus casei bacterium or (b) a Lactobacillus casei bacterium including one or more exogenous genes encoding a pyruvate decarboxylase and one or more exogenous genes encoding an alcohol dehydrogenase II, each of which is operably linked to a L. casei promoter that is not highly expressed in the stationary phase.

18. The method of claim 15, wherein the composition produced further comprises pyruvate, and wherein the amount of pyruvate remaining in the composition produced is significantly less than the amount of pyruvate in a composition produced by a Lactobacillus casei bacterium cultured on a substantially similar substrate for the same amount of time that is either (a) a wild-type Lactobacillus casei bacterium or (b) a Lactobacillus casei bacterium including one or more exogenous genes encoding a pyruvate decarboxylase and one or more exogenous genes encoding an alcohol dehydrogenase II, each of which is operably linked to a L. casei promoter that is not highly expressed in the stationary phase.

19. The method of claim 1, wherein the amount of ethanol in the composition produced is greater than 58% of the theoretical yield of ethanol, based on the amount of carbohydrate used.

20. The method of claim 18, wherein the amount of ethanol in the composition produced is from 79% to 89% of the theoretical yield of ethanol, based on the amount of carbohydrate used.
